(19) United States
(12) Patent Application Publication
(10) Pub. No.: US 2006/0052655 A1
(43) Pub. Date: Mar. 9, 2006

(54) Method and apparatus for modulating the effect of electro magnetic fields on the human body

(76) Inventor: Walter T. Myers SR., Tomball, TX (US)

Correspondence Address: NICK A NICHOLS, P O BOX 16399, SUGARLAND, TX, 774966399, US

(21) Appl. No.: 11/218,138

(22) Filed: Sep. 1, 2005

Related U.S. Application Data
(60) Provisional application No. 60/607,700, filed on Sep. 7, 2004.

(51) Int. Cl. A61N 2/00

(52) U.S. Cl. 600/9

(57) ABSTRACT

A waveform output apparatus comprises a circuit including a capacitor, resistors, and amplifiers, an LED and a coil connected to a power supply. The circuit is housed in a portable housing. When energized, the apparatus outputs waveforms that modulate EMF and ELF signals that may be absorbed by the human body.

### CROSS-REFERENCE TO RELATED APPLICATION

This application claims the benefit of U.S. Provisional Application Ser. No. 60/607,700, filed Sep. 7, 2004, which application is incorporated herein by reference.

### BACKGROUND OF THE INVENTION

1. Field of the Invention

The present invention relates to waveform output apparatus, particularly to waveform output apparatus for modulating the effect of electro magnetic fields (EMF) on the human body.

2. Description of the Related Art

Electromagnetic field or EMF is a broad term which includes electric fields created by electric charges, magnetic fields generated by electric charges in motion (i.e., electric current), and radiated fields such as television, radio, and microwaves. Electric and magnetic fields are generated by currents running through electric wires. A magnetic field exists in and around every magnetized substance and around every electric current. Electric fields are measured in units of volts per meter (V/m) and magnetic fields are measured in milli-Gauss (mG). The EMF is always strongest near the source and diminishes with increasing distance from the source. The energies from these fields have the ability to influence particles at great distances.

The energy that surrounds us is part of our environment. Each of us as power users live in electrical environments that naturally have very low electromagnetic fields. We are constantly exposed to various levels of EMF through the modern convenience of our daily life. For example, EMF is generated by electronic devices such as hair dryers, computers, monitors, television, and cellular telephones, just to name a few. Although electrical technology is a vital part of our daily existence, the health effects of EMF on biological tissue remains a very controversial issue—an issue requiring more research to determine safe or dangerous levels of EMF exposure.

The possible effects of EMF on the human body are also well known. EMF is believed to affect the iron necessary for healthy blood and stored in the brain; the permeability of the cell membrane of our nerves, blood vessels, skin, and other organs; and the intricate DNA of chromosomes. Every biological process throughout our bodies involves precisely choreographed movement of EMF-sensitive atoms, molecules and ions.

While it is not known if typical, everyday exposures to EMF affects human health, some studies suggest an EMF-illnesses connection. For example, some scientific studies have suggested a link between exposure to 60 hertz EMFs (the electric power in North America is 60 hertz alternating current) and specific types of cancer, primarily leukemia and brain cancer. While it is not known with certainty that exposure to EMFs in the everyday environment adversely affects health, it is also not at all clear whether such exposure to these invisible fields is safe in terms of human health.

The human brain produces electric fields that have been measured using electroencephalogram technology (EEG). Studies have shown that brain activity operates from 8 to 12 Hertz in the Alpha range and from 13 to 25 Hertz in the Beta range. These frequency ranges are associated with certain states of consciousness, such as alertness and sleep. It is also known that the human brain and body function in the milligauss magnetic field. The human body produce specific magnetic pulsations known as biomagnetic fields. Biomagnetic recordings, such as magnetocardiograms and magnetoencephalograms, are now complementing traditional recordings, such as the electrocardiogram and electroencephalogram, as diagnostic tools. Mapping the magnetic fields in the space around the body may often provide a more accurate indication of physiology and pathology than traditional electrical measurements.

In addition to health-related issues, electromagnetic interference has been identified as a problem by electronic manufacturers of products for both the consumer and business markets. Some common wireless devices, such as cellular telephones and two-way radios, are known to cause certain equipment to malfunction from the electromagnetic energy transmitted by the wireless device interfering with the sensitive circuitry. The use of cellular telephones, for example, is prohibited by most hospitals because of the electromagnetic interference that may affect hospital equipment. Some waveform output devices, as for example the devices described in U.S. Pat. No. 6,313,686, include an electromagnetic interference (EMI) canceling mechanism for controlling the amplitude of the waveform output by such devices.

U.S. Pat. No. 5,787,340, issued to Nokia Mobile Phones, discloses a communication device having a shield layer between an antenna and the user to reduce the electromagnetic irradiation to the user. The '340 patent states that “it has been suggested that modulated radio frequency radiation induces changes in the electrical status i.e. in the ion balance of nerve cells. A continuous localized exposure to radio frequency irradiation has been suggested to weaken myelin sheets of cells and to eventually lead to an impairment of hearing capability, vertigo, etc. It has been suggested that radio frequency irradiation may stimulate extra growth among supportive cells in the nerve system, which in the worst case it has been suggested could [lead] to a development of malignant tumor e.g. glioma from supportive cells. Although the consequences described above have not been scientifically verified, the uncertainty has some effects e.g. by reducing the speed of growth of the market of radiophones.”

EMFs are part of our daily environment and consequently have an effect on the human body, particularly the extremely low frequency (ELF) end of the electric and magnetic fields spectrum which may interfere with the human brain and body functions resulting in fatigue, muscle weakness, a decrease in concentration and an overall reduction in efficiency of brain and body activities.

It is desirable to have an apparatus that protects the human body from absorbing external EMF. It is desirable to intercept and/or cancel incoming EMF prior to being absorbed by the human body. It is an object of this invention to provide an apparatus that emits an electromagnetic pulse that minimizes or reduces the absorption of a range of external frequencies by the human body.

It is also desirable to have an apparatus to modulate, intercept or cancel EMF signals over a range of frequencies, and particularly in the 6-15 Hertz range.

### SUMMARY OF THE INVENTION

The present invention is a waveform output apparatus that emits an electromagnetic pulse that intercepts and/or cancels incoming EMF prior to being absorbed by the human body. The apparatus of the present invention intercepts or cancels incoming EMF and substantially eliminates the absorption of a range of external frequencies by the human body. In one embodiment of the present invention, the waveform output apparatus comprises a circuit, preferably an integrated circuit, including a capacitor, resistors, and amplifiers, an LED and a coil connected to a power supply. The circuit is housed in a portable housing. When energized, the apparatus outputs waveforms that modulate EMF signals within a specified frequency range.

The waveform output apparatus of the present invention emits alternating positive and negative electromagnetic pulses at a rapid rate, preferably at a rate of approximately two (2) bursts per second. In operation, the waveform emitted is a square wave with one pulse being positive and the next wave being negative. The positive and negative pulses are repeated continuously as rapid bursts, which in turn will cause the needle of a compass placed adjacent the waveform output apparatus to spin.

### BRIEF DESCRIPTION OF THE DRAWINGS

So that the manner in which the above recited features, advantages and objects of the present invention are attained can be understood in detail, a more particular description of the invention briefly summarized above, may be had by reference to the embodiments thereof which are illustrated in the appended drawings.

It is noted, however, that the appended drawings illustrate only typical embodiments of this invention and are therefore not to be considered limiting of its scope, for the invention may admit to other equally effective embodiments.

FIG. 1 is a perspective view of one embodiment of the waveform output apparatus of the present invention;

FIG. 2 is a schematic diagram of the waveform output apparatus of FIG. 1;

FIGS. 3 and 4 are diagrams illustrating the amplitude and energy redistribution of waveforms of equal amplitude and wavelength that are 180° out of phase;

FIG. 5 is a diagram showing the waveform of the output apparatus of an embodiment of the present invention versus time;

FIG. 6 is a diagram showing the waveform of the output apparatus of an embodiment of the present invention 180° out of phase with a sine wave; and

FIG. 7 is a schematic diagram of a second embodiment of the waveform output apparatus of the present invention.

### DETAILED DESCRIPTION OF A PREFERRED EMBODIMENT

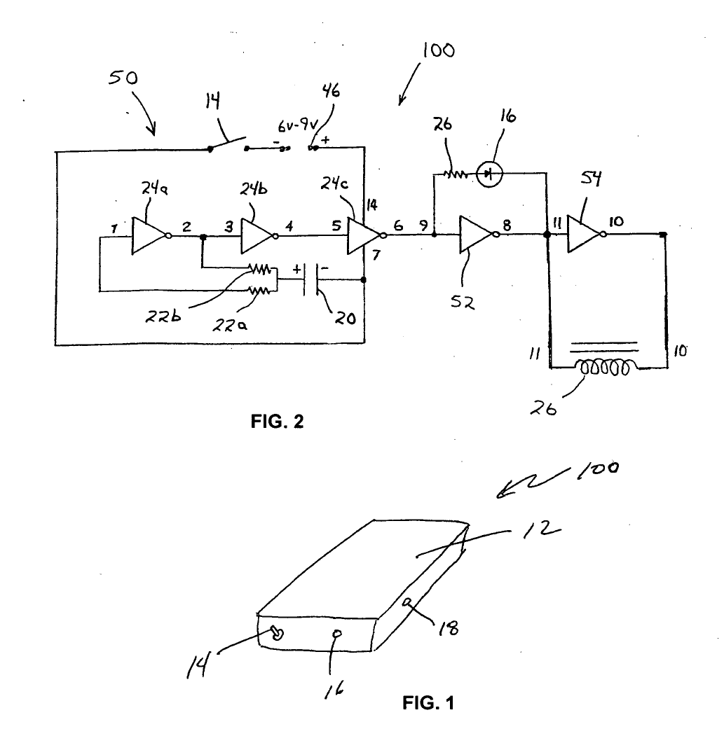

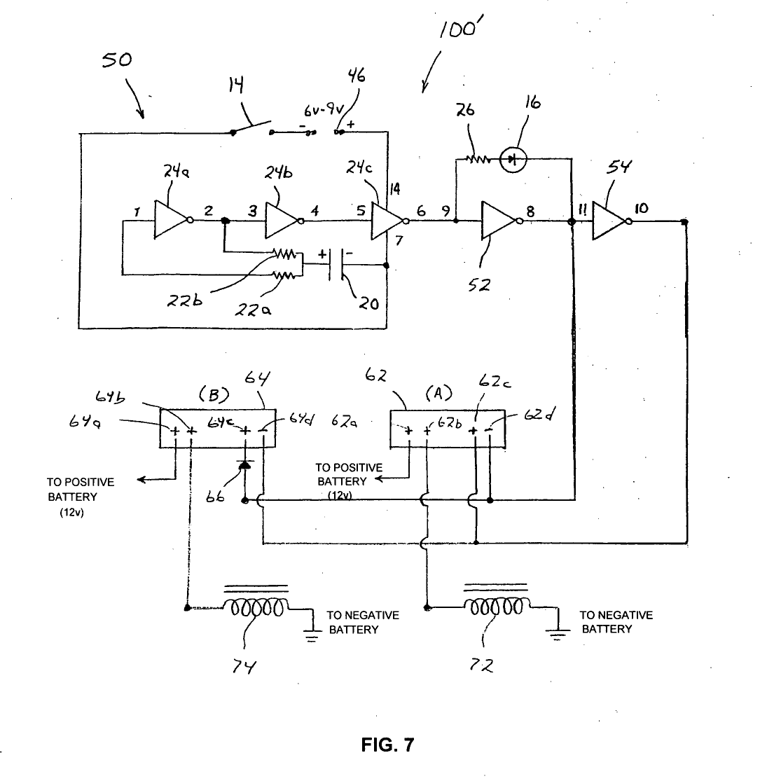

Referring to FIG. 1, a first embodiment of the waveform output apparatus of the present invention is generally identified by the reference numeral 100. The waveform output apparatus 100 comprises a housing 12 enclosing an electrical circuit arrangement, shown schematically in FIG. 2, that when energized emits an electromagnetic pulse. FIG. 7 is a schematic diagram of a second embodiment of the waveform output apparatus, generally designated as 100′. It is to be understood that the waveform output apparatus of the first and second embodiments, 100 and 100′ respectively, are identical in many respects. As further described below, the waveform output apparatus 100′ is a larger unit that provides a larger area of protection on a square footage basis than the smaller unit 100. It is to be understood, however, that the principles and operation of the apparatus 100 and 100′ are similar and the differences between the two apparatus will be explained in detail below. Unless stated otherwise, the discussion of the waveform output apparatus 100 also applies to the waveform output apparatus 100′.

Referring to FIG. 1, the waveform output apparatus 100 includes a housing 12 having an on-off switch 14 and a light emitting diode (LED) 16 mounted to the housing 12. The LED 16 is a visual indicator that the waveform output apparatus 100 is energized and operating. The housing 12 also includes an internal compartment (not shown in the drawings) for holding a battery 46 (FIG. 2) to power the waveform output apparatus 100. A power jack 18 is also provided for plugging the waveform output apparatus 100 to an AC power supply (not shown) if desired.

The first embodiment of the waveform output apparatus 100 shown in FIG. 1 may be dimensionally small, such as approximately the size of a deck of playing cards, and thus, will not occupy much surface space on a desk or the like. Alternatively, it may also be easily attached to a computer monitor, carried in a pocket or clipped on a belt, clothing or the like.

The waveform output apparatus 100 of the present invention emits alternating positive and negative electromagnetic bursts at a rapid rate, preferably at a rate of approximately two (2) bursts per second. In operation, the waveform emitted is a square wave with one pulse being positive and the next pulse being negative. The positive and negative pulses are repeated continuously as rapid bursts, which in turn will cause the needle of a compass placed adjacent the waveform output apparatus 100 to spin.

Referring now to FIG. 2, the waveform output apparatus 100 preferably includes an integrated circuit (IC) chip 50 mounted within the housing 12. The IC chip 50 includes a capacitor 20, two resistors 22a and 22b, and three “notted” amplifiers 24a, 24b and 24c. The two resistors 22a and 22b are coupled to the capacitor 20 which sets up a time chain to alternatingly pulse positive and negative.

The circuit of the waveform output apparatus 100 is generally completed by the LED 16, connected to a resistor 26 on its positive side, and a coil 26. The coil 26 preferably comprises an iron bar wound with magnetic wire. The circuit also includes fourth and fifth “notted” amplifiers 52 and 54, respectively.

Utilizing surface mounted technology (SMT) and integrated circuit (IC) technology in the preferred embodiments of the present invention, the power input to pin 14 in the schematic diagram of FIG. 2 is preferably in the range of +3 to +12 volts with SMT or preferably in the range of +5 to +12 volts with IC technology. When the switch 14 is closed, pin 7, which is always negative, activates the chip 50. Pins 1 and 2, with the resistors 22a and 22b connected in parallel to the positive side of the capacitor 20, create a square wave timing sequence. The amplification signal square wave pulses negative and positive. This generates alternating low and high outputs (negative and positive pulses) to pin 9. The “notted” amplifier 52 has input at pin 9 and output at pin 8 and the “notted” amplifier 54 has input at pin 11 and output at pin 10. When the output of amplifier 24c at pins 6 and 9 is high, the LED 16 is energized, pins 8 and 11 are low and pin 10 is high. When the output of amplifier 24c at pins 6 and 9 goes low, the LED is not energized, pins 8 and 11 are high and pin 10 is low. Pins 11 and 10, which are connected to the coil 26, pulse positive and negative every one-half second from the generated outputs from amplifiers 52 and 54, thus creating positive and negative cycles energizing the coil 26. The electromagnetic output of the coil 26 alternates between negative and positive electromagnetic bursts in the 60 to 80 milligauss range.

In use, the waveform output apparatus 100 of the present invention generates alternating negative and positive electromagnetic bursts that modulate EMF and ELF waveforms in various frequency ranges, including the 6-15 Hertz range, within a radius of approximately 6 to 10 feet of the apparatus 100. The positive and negative cycles of the coil 26 generate waveforms that are 180° out of phase with the EMF and ELF waveforms in the area of the apparatus 100 which results in interception of the incoming wave and substantial cancellation or modulation of the incoming wave.

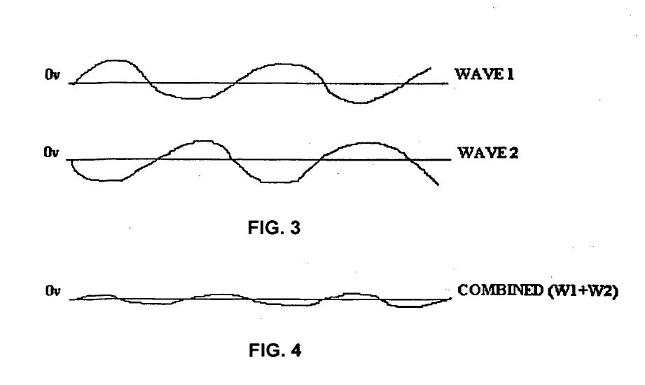

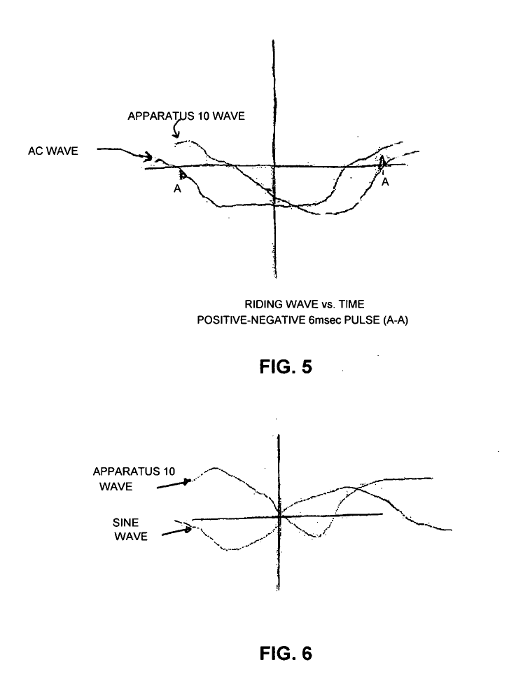

The human body absorbs waveforms of a variety of frequencies. The waveform output apparatus 100 of the present invention sends a waveform into space having a square wave output which attaches itself to the incoming wave. The square wave output is 180 degrees out of phase with the incoming wave and substantially cancels the incoming wave. With reference to FIG. 3, Wave 1 represents an incoming wave and Wave 2 represents a wave generated by the waveform output apparatus 100 which is 180° out of phase with the EMF and ELF waveforms in the area of the apparatus 100. When two waves of equal amplitude and wavelength are 180° out of phase as shown in FIG. 3, the result is a decrease of amplitude and energy redistribution in a new direction according to the Law of Conservation of Energy, as illustrated in FIG. 4. The resultant effect is that the absorption of external EMFs and ELFs by the human body is alleviated.

In a preferred embodiment of the waveform output apparatus 100 of the present invention, the resistors 22a and 22b each have a value preferably in the range of 22K to 47K ohms, and more preferably approximately 33 K ohms, the capacitor 20 has a value preferably in the range of 22 to 47 microfarads (mfd), and more preferably approximately 22 mfd, the amplifiers 24a, 24b, 24c, 52 and 54 are model no. CD40106BE, commercially available from electronics supply houses, and the resistor 26 has a value preferably in the range of 2.0 K to 3.9 K ohms, and more preferably approximately 3.9 K ohms. The electrical resistance of the coil 26 is preferably in the range of 200 to 500 ohms, and more preferably is approximately 383 ohms for the illustrated embodiment of the waveform output apparatus 100 shown in FIG. 2.

As shown in FIG. 7, the second embodiment of the waveform output apparatus 100′ is identical to the waveform output apparatus 100 with the exception of the coil 26. In the waveform output apparatus 100′, the coil 26 is replaced with first and second opto isolators, 62 and 64 respectively, and first and second coils, 72 and 74 respectively. The coils 72 and 74 are preferably solenoid coils. The opto isolators are an input-output optical device which allows the coils to pulse at a higher voltage—positively and negatively. This results in the waveform output apparatus 100′ being able to provide more power to the coils and cover a greater area.

Each opto isolator 62 and 64 includes four terminals, 62a-d and 64a-d respectively. The coil 72 is connected at one end to positive terminal 62b of the first opto isolator 62 and at the other coil end to the negative or ground of a battery. Similarly, the coil 74 is connected at one end to positive terminal 64b of the second opto isolator 64 and at the other coil end to the negative or ground of the battery. Opto isolators 62 and 64 have positive terminal 62a and 64a, respectively, connected to the positive of the battery. The battery is preferably a 12 volt battery.

Still referring to FIG. 7, pin 11 is connected to negative terminal 62d and positive terminal 64c of opto isolators 62 and 64, respectively. A diode 66 is included in the circuit adjacent the positive terminal 64c. Pin 10 is connected to positive terminal 62c and negative terminal 64d of opto isolators 62 and 64, respectively. With the configuration as shown, it is to be understood that when the coil is “on” or emitting a pulse, the other coil is “off.”

Referring to FIG. 7, when the pulse-generated output from amplifier 52 goes low at pin 8, pin 11 goes low. This results in low going in the first opto isolator at terminal 62d. At the same time, pin 10 of amplifier 54 goes high to the positive terminal 62c of the first opto isolator. Therefore, this action activates the first opto isolator (A) 62 and energizes coil 72, causing coil 72 to saturate.

Still referring to FIG. 7, the pulse-generated output from amplifier 52 at pin 8 and pin 11 then goes high and pin 10 goes low, which causes proper negative to pin 64d and proper positive to pin 64c of the second opto isolator 64. Therefore, this action activates the second opto isolator (B) 64 and energizes coil 74, causing coil 74 to saturate. Because the pulse is every 0.5 seconds, the collapsing field from one coil is opposite the saturating field of the other coil.

In the preferred embodiment of the waveform output apparatus 100′, the opto isolators are commercially available as Model No. ODCS 5. Preferably, the electrical resistance of each of the coils 72 and 74 is in the range of 20 to 150 ohms, and more preferably is approximately 83.6 ohms for the illustrated waveform output apparatus 100′.

The waveform output apparatus 100′ described above is intended to provide protection over an area of 5,000 square feet or greater.

In the preferred embodiments of the present invention as described above, an integrated circuit chip 50 is utilized, although the present invention may alternatively be accomplished using transistors. Thus, it is to be understood that while the present invention has been described using IC componentry, it could also be accomplished using transistor-transistor logic (TTL). However, it is believed that IC componentry yields better output and a cleaner pulse rhythm.

While a preferred embodiment of the invention has been shown and described, other and further embodiments of the invention may be devised without departing from the basic scope thereof, and the scope thereof is determined by the claims which follow.

1. An apparatus for modulating EMF and ELF signals within a frequency range having an effect on the human body, comprising a waveform output unit for outputting a waveform 180° out of phase with the EMF and ELF signals.

2. The apparatus of claim 1 wherein said waveform output unit includes an integrated circuit component and an inductor.

3. The apparatus of claim 2 wherein said inductor is a coil and wherein the electrical resistance of said coil is in the range of 200 to 500 ohms.

4. The apparatus of claim 3 wherein the electrical resistance of said coil is approximately 383 ohms.

5. The apparatus of claim 2 including an LED indicator.

6. The apparatus of claim 3 wherein said frequency range is 6-15 hertz.

7. The apparatus of claim 1 wherein said waveform output unit generates alternating negative and positive electromagnetic bursts in a frequency range of 6-15 hertz.

8. The apparatus of claim 1 wherein said waveform output unit generates a waveform having a square wave output.

9. The apparatus of claim 1 wherein said waveform output unit includes an integrated circuit, first and second opto isolators, and first and second coils.

10. The apparatus of claim 9 wherein said first and second coils are solenoid coils.

11. The apparatus of claim 9 wherein the electrical resistance of said first and second coils is in the range of 20 to 150 ohms.

12. The apparatus of claim 11 wherein the electrical resistance of said first and second coils is approximately 83.6 ohms.
